(19) United States
(12) Patent Application Publication
(10) Pub. No.: US 2021/0335080 A1
(43) Pub. Date: Oct. 28, 2021

(54) MOBILE PHARMACEUTICAL DISPENSING DEVICES

(71) Applicant: Tech Pharmacy Services, LLC, Fort Lee, NJ (US)

(72) Inventors: Omer EINAV, Kfar-Monash (IL); Doron SHABANOV, Tzur-Yigal (IL); Tamir BEN DAVID, Tel-Aviv (IL); Anthony Joseph SPERO, Queensbury, NY (US); Eyal LIVSCHITZ, Givat Shmuel (IL); Thomas A. MCKINNEY, Boonton, NJ (US); Moshe LIBERMAN, Yehud (IL)

(73) Assignee: Tech Pharmacy Services, LLC, Fort Lee, NJ (US)

(21) Appl. No.: 17/237,169

(22) Filed: Apr. 22, 2021

Related U.S. Application Data
(60) Provisional application No. 63/014,145, filed on Apr. 23, 2020.

(51) Int. Cl. G07F 17/00; G07F 9/00; G07F 11/16; G07F 11/38

(52) U.S. Cl. CPC G07F 17/0092; G07F 9/001; G07F 11/38; G07F 11/1653; G07F 9/009

(57) ABSTRACT

The present invention relates to pharmaceutical dispensing vehicles and/or mobile pharmaceutical dispensing machines, comprising: a vehicle and/or a frame comprising an inside and an outside; one or more pharmaceutical dispensing modules configured to perform parts of a pharmaceutical dispensing process, said one or more modules located inside said vehicle and/or frame; one or more delivery windows facing the outside of said vehicle and or frame, said one or more delivery windows are in communication with said one or more pharmaceutical dispensing modules and are configured to receive one or more pharmaceuticals ready to be dispensed; one or more user interfaces facing the outside of said vehicle and/or frame, said one or more user interfaces are in communication with said one or more pharmaceutical dispensing modules and are configured to receive one or more input related to a pharmaceutical dispensing request from at least one user.

### RELATED APPLICATIONS

This application claims the benefit of priority under 35 USC § 119(e) of U.S. Provisional Patent Application No. 63/014,145 filed on Apr. 23, 2020, the contents of which are incorporated by reference as if fully set forth herein in their entirety.

### FIELD AND BACKGROUND OF THE INVENTION

The present invention, in some embodiments thereof, relates to a pharmaceutical dispensing machine and, more particularly, but not exclusively, to a mobile pharmaceutical dispensing machine.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A mobile pharmaceutical dispensing machine, comprising:

a. an external frame comprising an inside and an outside;

b. one or more pharmaceutical dispensing modules located in said inside of said external frame and configured to perform parts of a pharmaceutical dispensing process of a pharmaceutical dispensing request from at least one user, said pharmaceutical dispensing process comprising moving one or more items from general containers into one or more personalized pharmaceutical dispensing containers;

c. one or more delivery windows facing said outside of said external frame and configured to receive and allow delivery of said one or more personalized pharmaceutical dispensing containers from said one or more pharmaceutical dispensing modules;

d. one or more user interfaces in communication with said one or more pharmaceutical dispensing modules and configured to receive one or more inputs related to said pharmaceutical dispensing request from said at least one user.

Example 2. The mobile pharmaceutical dispensing machine of example 1, wherein said one or more modules are selected from the group consisting of: at least one pharmaceutical array module, at least one storage module, at least one pharmaceutical operational modules section, at least one user interface module, at least one communication module and at least one mechanical arm module.

Example 3. The mobile pharmaceutical dispensing machine of example 1 or 2, wherein said at least one user interface module is accessed by said at least one user using at least one mobile device.

Example 4. The mobile pharmaceutical dispensing machine of any one of examples 1-3, wherein said at least one user interface module comprises at least one user interface panel facing said outside of said mobile pharmaceutical dispensing machine and located at a distance from about 0.9 meters to about 1.7 meters from the ground on which said mobile pharmaceutical dispensing machine is standing.

Example 5. The mobile pharmaceutical dispensing machine of any one of examples 1-4, wherein said at least one user interface module comprises identification hardware and software configured to recognize said at least one user trying to access the services of said mobile pharmaceutical dispensing machine.

Example 6. The mobile pharmaceutical dispensing machine of any one of examples 1-5, wherein said at least one user interface panel comprises self-sterilization equipment.

Example 7. The mobile pharmaceutical dispensing machine of any one of examples 1-6, wherein said one or more delivery windows are located at a distance from about 0.9 meters to about 1.7 meters from the ground on which said mobile pharmaceutical dispensing machine.

Example 8. The mobile pharmaceutical dispensing machine of any one of examples 1-7, wherein said one or more delivery windows comprises self-sterilization equipment.

Example 9. The mobile pharmaceutical dispensing machine of any one of examples 1-8, wherein said one or more modules are configured to be independently dismounted from said mobile pharmaceutical dispensing machine; and wherein dismounting said one or more modules does not interrupt said parts of said pharmaceutical dispensing process.

Example 10. The mobile pharmaceutical dispensing machine of any one of examples 1-9, wherein said at least one pharmaceutical array module is configured to store pharmaceuticals and non-pharmaceuticals.

Example 11. The mobile pharmaceutical dispensing machine of any one of examples 1-10, wherein said one or more user interfaces comprises one or more two-way communication systems comprising one or more screens, one or more cameras and one or more microphones, configured to allow communication between said at least one user and an authorized personnel.

Example 12. The mobile pharmaceutical dispensing machine of any one of examples 1-11, further comprising at least one written prescription processing system configured to process written prescriptions presented by said at least one user.

Example 13. The mobile pharmaceutical dispensing machine of any one of examples 1-12, wherein said one or more user interfaces are further configured to display instructions related to said pharmaceuticals and said non-pharmaceuticals.

Example 14. The mobile pharmaceutical dispensing machine of any one of examples 1-13, wherein said mobile pharmaceutical dispensing machine further comprises one or more wheels and at least one motor in communication with said one or more wheels and configured to actively rotate said one or more wheels.

Example 15. The mobile pharmaceutical dispensing machine of any one of examples 1-14, wherein said mobile pharmaceutical dispensing machine is a motor vehicle.

Example 16. The mobile pharmaceutical dispensing machine of any one of examples 1-15, wherein said mobile pharmaceutical dispensing machine is an autonomous mobile pharmaceutical dispensing machine and further comprises location and navigation systems.

Example 17. A method for providing a mobile pharmaceutical dispensing machine to an event, comprising:

a. receiving request for said pharmaceutical dispensing vehicle and/or said mobile pharmaceutical dispensing machine;

b. assessing whether sending said mobile pharmaceutical dispensing machine;

c. assessing one or more of said parameters of said event;

d. equipping said mobile pharmaceutical dispensing machine according to the results of said assessments;

e. deploying said mobile pharmaceutical dispensing machine to a location of said event.

Example 18. The method according to example 17, wherein said assessing one or more parameters of said event comprises one or more of assessing type of event, assessing expected population in event, assessing said location of event.

Example 19. The method according to example 17 or 18, wherein said type of event is an emergency event.

Example 20. The method according to any one of examples 17-19, wherein said emergency event is a viral pandemic event.

Example 21. The method according to any one of examples 17-20, wherein said viral pandemic event is a COVID-19 pandemic event.

Example 22. The method according to any one of examples 17-21, wherein said type of event is a scheduled event.

Example 23. The method according to any one of examples 17-22, wherein said expected population comprises one or more of: elderly population, young population, healthy population, sick population, COVID-19 positive population, quarantined population, newborn population, middle age population and mixed population.

Example 24. The method according to any one of examples 17-23, wherein said location comprises one or more of: public location, private location, a concert, a stadium, a hotel, a hotel used for quarantined patients, a hotel used for COVID-19 quarantined patients, a park, middle of the street, a location of an emergency event and a location of an accident.

Example 25. A method for setting a pick-up location of one or more items by a user from a mobile pharmaceutical dispensing machine, comprising:

a. receiving from said user a request to provide possible pick-up locations for said one or more items from a mobile pharmaceutical dispensing machine;

b. assessing required time to complete request according to requested items;

c. providing said user a list of possible pick-up locations according to results of (b);

d. receiving from user a chosen location to pick-up;

e. confirming to user the pick-up location;

f. providing said one or more items to a mobile pharmaceutical dispensing machine which will be at the chosen pick-up location.

Example 26. A method for setting an alternative entity to pick-up one or more items from a mobile pharmaceutical dispensing machine located at a specific location, comprising:

a. receiving from a user a request to provide permission to an alternative entity to physically pick-up said one or more items from said mobile pharmaceutical dispensing machine located at said specific location;

b. requesting from said user personal identification data of said alternative entity;

c. receiving personal identification data of alternative entity;

d. updating personal identification data of alternative entity;

e. requesting method of identification of the alternative entity at time of pick-up;

f. updating system of chosen method of identification.

Example 27. A pharmaceutical dispensing vehicle, comprising:

a. a vehicle comprising an inside and an outside;

b. one or more pharmaceutical dispensing modules configured to perform parts of a pharmaceutical dispensing process, said one or more modules located inside said vehicle;

c. one or more delivery windows facing the outside of said vehicle, said one or more delivery windows are in communication with said one or more pharmaceutical dispensing modules and are configured to receive one or more pharmaceuticals ready to be dispensed;

d. one or more user interfaces, said one or more user interfaces are in communication with said one or more pharmaceutical dispensing modules and are configured to receive one or more input related to a pharmaceutical dispensing request from at least one user.

Example 28. The pharmaceutical dispensing vehicle of example 27, wherein said one or more modules are selected from the group consisting of: at least one pharmaceutical array module, at least one storage module, at least one pharmaceutical operational modules section, at least one user interface module, at least one communication module and at least one mechanical arm module.

Example 29. The pharmaceutical dispensing vehicle of example 27 or 28, wherein said at least one user interface module is accessed by said at least one user using at least one mobile device.

Example 30. The pharmaceutical dispensing vehicle of any one of examples 27-29, wherein said at least one user interface module comprises at least one user interface panel facing said outside of said pharmaceutical dispensing vehicle and located at a distance from about 0.9 meters to about 1.7 meters from the ground on which said vehicle is standing.

Example 31. The pharmaceutical dispensing vehicle of any one of examples 27-30, wherein said at least one user interface module comprises identification hardware and software configured to recognize patients and/or dedicated personnel trying to access the services of said pharmaceutical dispensing vehicle.

Example 32. The pharmaceutical dispensing vehicle of any one of examples 27-31, wherein said at least one user interface panel comprises self-sterilization equipment.

Example 33. The pharmaceutical dispensing vehicle of any one of examples 27-32, wherein said at least one user interface module comprises at least one delivery window in communication with said outside of said pharmaceutical dispensing vehicle and located at a distance from about 0.9 meters to about 1.7 meters from the ground on which said vehicle is standing.

Example 34. The pharmaceutical dispensing vehicle of any one of examples 27-33, wherein said at least one delivery window comprises self-sterilization equipment.

Example 35. The pharmaceutical dispensing vehicle of any one of examples 27-34, wherein said one or more modules are configured to be independently dismounted from said pharmaceutical dispensing vehicle; and wherein dismounting said one or more modules does not interrupt said parts of said pharmaceutical dispensing process.

Example 36. The pharmaceutical dispensing vehicle of any one of examples 27-35, wherein said at least one pharmaceutical array module is configured to store pharmaceuticals and non-pharmaceuticals.

Example 37. The pharmaceutical dispensing vehicle of any one of examples 27-36, wherein said one or more user interfaces comprises one or more two-way communication system comprising one or more screens, one or more cameras and one or more microphones, configured to allow communication between said at least one user and an authorized personnel.

Example 38. The pharmaceutical dispensing vehicle of any one of examples 27-37, further comprising at least one written prescription processing system configured to process written prescriptions presented by said at least one user.

Example 39. The pharmaceutical dispensing vehicle of any one of examples 27-38, wherein said one or more user interfaces are further configured to display instructions related to said pharmaceuticals and said non-pharmaceuticals.

Example 40. The pharmaceutical dispensing vehicle of any one of examples 27-39, wherein said pharmaceutical dispensing vehicle further comprises one or more wheels and at least one motor in communication with said one or more wheels and configured to actively rotate the one or more wheels.

Example 41. The pharmaceutical dispensing vehicle of any one of examples 27-40, wherein said pharmaceutical dispensing vehicle is an autonomous pharmaceutical dispensing vehicle and further comprises location and navigation systems.

Example 42. A method for deploying an autonomous pharmaceutical dispensing machine, comprising:

a. receiving from system one or more locations and/or one or more schedules where and/or where mobile pharmaceutical dispensing machine is needed;

b. assessing inventory of mobile pharmaceutical dispensing machine;

c. assessing possible routes according to locations and schedules in order to meet dispensing requirements; and

d. deploying mobile pharmaceutical dispensing machine according to result of (c).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as receiving pharmaceutical prescriptions, preparing pharmaceuticals to be dispensed, dispensing pharmaceuticals, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an exemplary pharmaceutical dispensing system, according to some embodiments of the invention;

FIG. 2 is an exemplary pharmaceutical dispensing machine, according to some embodiments of the invention;

FIGS. 3a-b are two exemplary mobile pharmaceutical dispensing machines, according to some embodiments of the invention;

FIG. 4 is a flowchart of an exemplary method of deployment of a pharmaceutical dispensing machine/vehicle for a scheduled event, according to some embodiments of the invention;

FIG. 5 is a flowchart of an exemplary method of interaction between the deployable pharmaceutical dispensing system and a user, according to some embodiments of the invention;

FIG. 6 is a flowchart of an exemplary method of interaction between the deployable pharmaceutical dispensing system and a user, according to some embodiments of the invention; and

FIG. 7 is a flowchart of an exemplary method of deployment of an autonomous mobile pharmaceutical dispensing machine, according to some embodiments of the invention.

### DESCRIPTION OF SPECIFIC EMBODIMENTS OF THE INVENTION

The present invention, in some embodiments thereof, relates to a pharmaceutical dispensing machine and, more particularly, but not exclusively, to a mobile pharmaceutical dispensing machine.

### Overview

An aspect of some embodiments of the invention relates to providing mobile pharmaceutical and/or non-pharmaceutical but medical related dispensing services to either scheduled events and/or emergency events. In some embodiments, the pharmaceuticals and/or non-pharmaceuticals but medical related are chosen according to the parameters of the event, for example, type of event (emergency or scheduled event), expected population in the event (young, elderly, mixed) and location of the event. In some embodiments, scheduled events can also be a single user that requested to pick-up a certain pharmaceutical and/or non-pharmaceutical.

An aspect of some embodiments of the invention relates to preparing pharmaceutical array modules according to topics and keeping them in storage until they are required. In some embodiments, topics can be scheduled events, for example concerts or sport events, and/or topics can be emergency events, for example accidents, terrorists attacks, shooting, etc.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

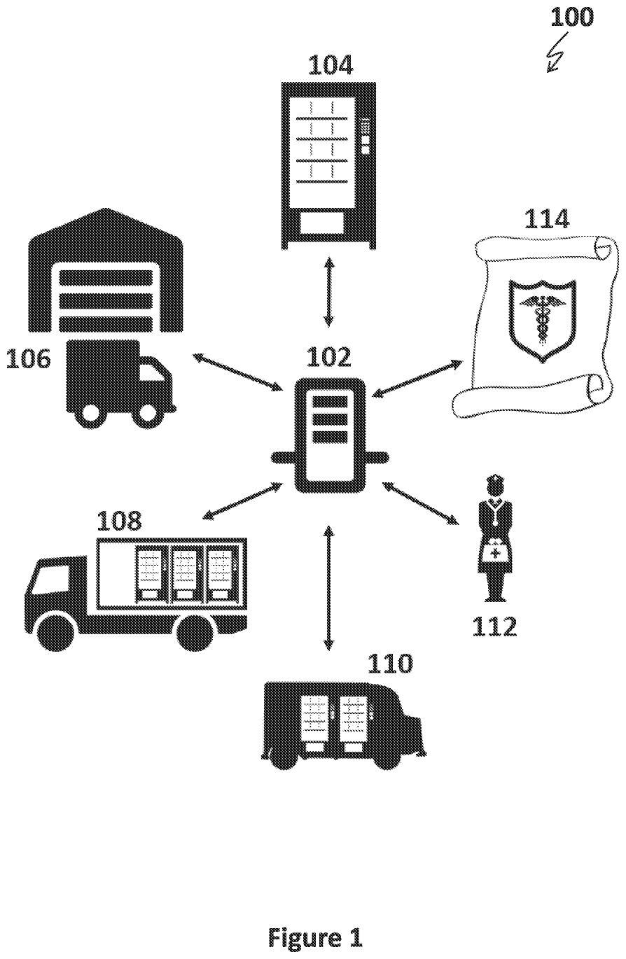

Referring now to the drawings, FIG. 1 illustrates an exemplary pharmaceutical dispensing system, according to some embodiments of the invention. In some embodiments, the pharmaceutical dispensing system 100 comprises one or more central servers 102, one or more pharmaceutical dispensing machines 104, one or more warehouses 106, one or more mobile pharmaceutical dispensing machines 108, one or more pharmaceutical dispensing vehicles 110, one or more dedicated medical personnel 112 (see below) and one or more medical insurance companies 114. In some embodiments, there is an exchange of information between the central server 102 and the other units of the system (pharmaceutical dispensing machines 104, warehouses 106, mobile pharmaceutical dispensing machines 108, pharmaceutical dispensing vehicles 110, dedicated medical personnel 112 and insurance companies 114), for example instructions, coordination of interactions between units of the system, prescriptions, pharmaceutical inventories, medical information, authorizations, technical status of the machines and/or technical assistance.

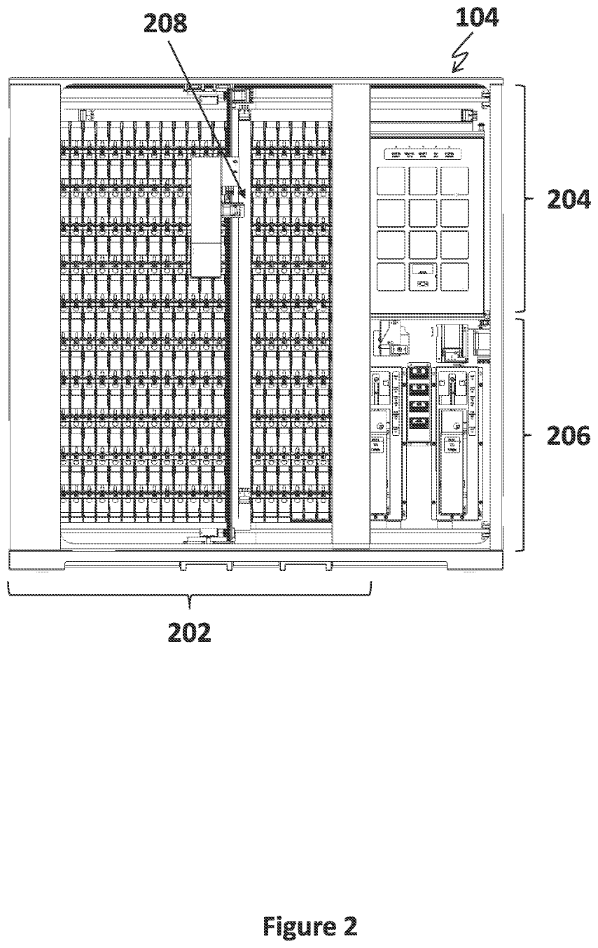

Referring now to the drawings, FIG. 2 illustrates an exemplary pharmaceutical dispensing machine 104, according to some embodiments of the invention. In some embodiments, the pharmaceutical dispensing machine comprises one or more modules selected from the group consisting of: at least one pharmaceutical array module 202, at least one pharmaceutical tote module and/or at least one storage module 204, at least one pharmaceutical operational modules section 206 and at least one mechanical arm module 208.

In some embodiments, the pharmaceutical dispensing machine 104 comprises at least one backup and/or redundant software and/or hardware. In some embodiments, said at least one backup and/or redundant software and/or hardware are located inside the same pharmaceutical dispensing machine.

The meaning of “located inside the same pharmaceutical dispensing machine” refers hereinafter to a single machine comprising an external envelope and/or frame that includes all components required for the pharmaceutical dispensing process, which, in some embodiments, also include backup and/or redundant software and/or hardware. It should be understood that two identical machines located one near the other with capabilities of transferring materials from one to the other, differ from the notion of “a pharmaceutical dispensing machine comprising at least one backup and/or redundant software and/or hardware located inside the same pharmaceutical dispensing machine”. The first disclose two independent machines located one near the other and connected by certain operational hardware, while the other disclose a single machine having a plurality of hardware inside the same external envelope, some of them identical or similar to each other and configured to perform the same and/or similar tasks.

In some embodiments, the pharmaceutical dispensing machine comprises at least one module. In some embodiments, the pharmaceutical dispensing machine comprises a plurality of modules. In some embodiments, said at least one module comprises at least one backup and/or redundant hardware and/or module adapted to perform the same and/or similar and/or different functions. In some embodiments, said at least one module and said at least one backup and/or redundant hardware and/or module perform tasks at the same time. In some embodiments, said at least one module comprises at least one backup and/or redundant hardware and/or module are located inside the same pharmaceutical dispensing machine.

In some embodiments, said at least one module comprises at least one unit. In some embodiments, said at least one module comprises a plurality of units. In some embodiments, said at least one unit comprises at least one backup and/or redundant hardware and/or unit adapted to perform the same and/or similar and/or different functions. In some embodiments, said at least one unit and said at least one backup and/or redundant hardware and/or unit perform tasks at the same time. In some embodiments, said at least one unit comprises at least one backup and/or redundant hardware and/or unit are located inside the same pharmaceutical dispensing machine.

In some embodiments, said at least one unit comprises at least one element. In some embodiments, said at least one unit comprises a plurality of elements. In some embodiments, said at least one element comprises at least one backup and/or redundant hardware and/or element adapted to perform the same and/or similar and/or different functions. In some embodiments, said at least one element and said at least one backup and/or redundant hardware and/or element perform tasks at the same time. In some embodiments, said at least one element comprises at least one backup and/or redundant hardware and/or element are located inside the same pharmaceutical dispensing machine.

In some embodiments, the modules, the units and the elements, comprise at least one dedicated sensor adapted to monitor the functioning and/or the performance and/or the demands of the modules/units/elements. In some embodiments, the at least one dedicated sensor is activated remotely by a user via a dedicated server.

In some embodiments, verification on functionality of hardware, updated software, content (e.g. pharmaceuticals), consumables (e.g. envelopes) is performed on the modules, the units and the elements. In some embodiments, a user and/or a technician at the site of the pharmaceutical dispensing machine perform the verification. In some embodiments, a user and/or a technician perform the verification remotely.

In some embodiments, the pharmaceutical dispensing machine comprising backup and/or redundant hardware and/or modules, also comprises at least two pharmaceutical array modules, at least two pharmaceutical tote modules and/or storage modules, at least two pharmaceutical control modules and at least two mechanical arm modules. In some embodiments, said backup and/or redundant hardware and/or modules are located inside the same pharmaceutical dispensing machine as the modules.

In some embodiments, only one module is operated while the backup and/or redundant hardware and/or module is inactive. In some embodiments, both the main module and the backup and/or redundant hardware and/or module are operated simultaneously.

In some embodiments, the modules, either main module or backup and/or redundant hardware and/or module can be operated independently to other hardware and/or modules. For example, the two pharmaceutical array modules can be operated while only one pharmaceutical tote module and/or storage module is operated. Similarly, the two pharmaceutical array modules and the two pharmaceutical tote and/or storage modules are operated while only one pharmaceutical operational module section is operated.

In some embodiments, the modules are designed to be an independent part of the pharmaceutical dispensing machine. For example, the storage module 204 can be disconnected (dismounted) from the frame of the pharmaceutical dispensing machine completely, without affecting the rest of the modules. In some embodiments, each module (202, 204, 206 and 208) can be extracted from the frame of the pharmaceutical dispensing machine. In some embodiments, each module (202, 204, 206 and 208) can be extracted from the frame of the pharmaceutical dispensing machine without the need to stop the operation of the machine.

In some embodiments, the pharmaceutical dispensing machine is configured to allow access to a technician to the internal modules of the pharmaceutical dispensing machine without the need to stop the operations of the pharmaceutical dispensing machine. In some embodiments, this configuration includes, for example, allowing access to the technician from the back side and/or the front side of the pharmaceutical dispensing machine while leaving the front side and/or the back side of the pharmaceutical dispensing machine free and undisturbed access for normal operations, for example, receiving the ready-to-be dispensed containers comprising the pharmaceuticals.

In some embodiments, this configuration is potentially advantageous during repairs and/or maintenance. In some embodiments, the time required to bring the pharmaceutical dispensing machine to normal functioning is very short (e.g. minutes). In some embodiments, the technician can pull out a module and then insert a new one at its place. In some embodiments, the time to replace either a module, a unit or an element from said pharmaceutical dispensing device is from about 1 minute to about 10 minutes. Optionally from about 5 minutes to about 30 minutes. Optionally from about 10 minutes to about 60 minutes.

### Exemplary Pharmaceutical Array Module 202

In some embodiments, the pharmaceutical array module 202 is responsible for the storage of a variety of pharmaceuticals and/or non-pharmaceuticals. In some embodiments, the pharmaceutical array module 202 comprises a plurality of drug units, which each further comprise a plurality of drug elements, in which pharmaceuticals are stored. While the term “drug unit” is used, it should be understood that the unit may include also non-drug contents (see below).

In some embodiments, the number of drug units in the pharmaceutical array module is from about 1 to about 500. Optionally, the number of drug units in the pharmaceutical array module is from about 4 to about 300. Optionally, the number of drug units in the pharmaceutical array module 202 is from about 10 to about 200. In some embodiments, each drug unit comprises a backup and/or redundant drug unit adapted to include the same drug elements. In some embodiments, said backup and/or redundant drug unit are located inside the same pharmaceutical dispensing machine as the drug units.

In some embodiments, each drug unit is designed to be an independent part of the pharmaceutical array module 202. For example, the drug unit can be disconnected (dismounted) from the frame of the pharmaceutical array module 202, completely, without affecting, for example, the correct functioning of the drug units. In some embodiments, a drug unit is adapted to include a specific number of drug elements within. While the term “drug element” is used, it should be understood that the element may include also non-drug contents (see below). In some embodiments, the number of drug elements within a drug unit is from about 2 to about 200. Optionally, is from about 5 to about 150. Optionally, is from about 10 to 100. In some embodiments, each row of drug unit comprises a plurality of drug subunits arranged one after the other.

In some embodiments, a drug unit comprises a backup and/or redundant drug unit adapted to perform the same function. In some embodiments, a drug subunit comprises a backup and/or redundant drug subunit adapted to perform the same function. In some embodiments, said backup and/or redundant drug subunit are located inside the same pharmaceutical dispensing machine as the drug subunits.

In some embodiments, the drug unit can be disconnected (dismounted) from the frame of the pharmaceutical array module 202. In some embodiments, this configuration is potentially advantageous during repairs and/or maintenance. In some embodiments, the time required to bring the pharmaceutical dispensing machine to normal functioning is very short (e.g. minutes). In some embodiments, the technician can pull out a unit and then insert a new one at its place. In some embodiments, during repairs and/or maintenance a backup and/or redundant drug unit and/or drug subunit, optionally and/or additionally located inside the same pharmaceutical dispensing machine and/or the same pharmaceutical array module and/or the same drug unit, performs the function of the drug unit and/or drug subunit that is being repaired and/or maintained.

### Exemplary Drug Unit/Drug Subunit

In some embodiments, the drug units/subunits are adapted to include a plurality of drug elements within. In some embodiments, the number of drug elements within a drug subunit is from about 2 to about 20. Optionally, is from about 5 to about 15. Optionally, is from about 7 to 10. In some embodiments, the drug units/subunits comprise an adapter configured to receive and lock in place a drug element. In some embodiments, pharmaceuticals are stored in the drug elements. In some embodiments, each drug element stores a different pharmaceutical. Optionally, several drug elements store the same kind of pharmaceuticals.

In some embodiments, the drug units/subunits can have a horizontal orientation with respect to the pharmaceutical array module 202. In some embodiments, the drug units/subunits can have a vertical orientation with respect to the pharmaceutical array module 202.

In some embodiments, the adapters are configured to provide the drug elements with an angle α in relation to the wall of the drug unit/subunit on which they are connected. In some embodiments, the angle α is from about 10 degrees to about 90 degrees. Optionally, the angle α is from about 20 degrees to about 60 degrees. Optionally, the angle α is from about 30 degrees to about 50 degrees. Optionally, the angle α is 30 degrees or 45 degrees or 60 degrees.

In some embodiments, each drug element comprises a backup and/or redundant drug element including the same type of pharmaceuticals. In some embodiments, said backup and/or redundant drug element are located inside the same pharmaceutical dispensing machine as the drug elements.

In some embodiments, each drug element is designed to be an independent part of the drug unit/subunit. In some embodiments, the drug element can be disconnected (dismounted) from drug unit. For example, the drug element can be disconnected (dismounted) from the frame of the drug unit, completely, without affecting, for example, the correct functioning of the drug elements.

In some embodiments, this configuration is potentially advantageous during repairs and/or maintenance. In some embodiments, the time required to bring the pharmaceutical dispensing machine to normal functioning is very short (e.g. minutes). In some embodiments, the technician can pull out a unit and then insert a new one at its place. In some embodiments, during repairs and/or maintenance a backup and/or redundant drug element, optionally and/or additionally located inside the same pharmaceutical dispensing machine and/or the same drug unit and performs the function of the drug element that is being repaired and/or maintained.

### Exemplary Drug Element

In some embodiments, each drug element comprises two main parts: a cartridge and a container. In some embodiments, the cartridge and the container can be separated from each other. In some embodiments, the cartridge is filled with single pharmaceutical pills. In some embodiments, after the cartridge is connected to the container, at the first use, a component in the mechanical arm module, engages button, which releases a flap, located at the bottom of the cartridge, thereby releasing the pharmaceutical pills into the container. In some embodiments, the pill located in the cartridge are released only when pills located in the container are finished. In some embodiments, the pills located in the cartridge are used as backup pills for the pills located in the container. In some embodiments, the cartridge comprises a tag (or a barcode or a qcode) at the top, comprising the relevant information regarding the pharmaceutical inside the cartridge. In some embodiments, the container comprises a base configured to interconnect with the adapter of the drug unit. In some embodiments, the container comprises an electronic tag, which includes the information related to the pharmaceuticals in the drug element. In some embodiments, the electronic tag is an RFID tag. Optionally, the electronic tag is a data dot, a barcode or qcode. In some embodiments, the container comprises a probe assembly comprising a cap with an adaptor configured to interconnect with a part of the mechanical arm module and a probe adapted to engage a single pharmaceutical pill (see below pill engagement mechanism). In some embodiments, the container optionally comprises a pill dam adapted to partially hold and separate the single pharmaceutical pills from the zone of the straw of the probe. In some embodiments, this configuration is potentially advantageous to facilitate the engagement with a single pharmaceutical pill.

In some embodiments, the cartridge and/or the container can be replaced without discarding the other.

In some embodiments, the machine comprises a plurality of spare probes in case a probe wears out and/or malfunctions. In some embodiments, a variety of designs of probes are used in the machine, for example, adapted to engage different types of pills. For example, the distal end of the probe, which engages the pills, can be designed with a circular geometry adapted to engage circular pills. Optionally, bigger probes are adapted to engage bigger pills.

In some embodiments, the drug elements may comprise different number of pills, different size of pills and/or different weight of pills.

### Exemplary Mechanical Arm Module

In some embodiments, the mechanical arm module 208 comprises two horizontal rails on which a vertical axle runs horizontally (right and left, left and right) by means of a motor. In some embodiments, the vertical axle moves horizontally as much as the length of the two horizontal axles. In some embodiments, the mechanical arm module 208 further comprises a pharmaceutical pill engagement mechanism that runs vertically (up and down, down and up) on the vertical axle by means of a motor. In some embodiments, the pharmaceutical pill engagement mechanism moves vertically as much as the length of the vertical axle.

In some embodiments, the mechanical arm module 208 can be disconnected (dismounted) from the frame of the pharmaceutical dispensing machine without the need to dismount and/or disassemble the other modules. Optionally, only one of the two rails is used. In these cases, the left right motion mechanism that provides the motion is either hanging from the top rail or standing from the bottom.

In some embodiments, the pharmaceutical dispensing machine 104 comprises two mechanical arms modules 208 in the same pharmaceutical dispensing machine. In some embodiments, the mechanical arm modules are located parallel to one another, thereby allowing both mechanical arm modules to work in parallel without interfering one with the activity of the other. In some embodiments, one of the two mechanical arm modules 208 operates while the other is not being operated. In some embodiments, both mechanical arm modules are operated at the same time. In some embodiments, during repairs and/or maintenance one mechanical arm module located inside the same pharmaceutical dispensing machine performs the function of the other mechanical arm module that is being repaired and/or maintained.

### Exemplary Pharmaceutical Tote Module and/or Storage Module

In some embodiments, after the envelopes and/or containers are marked, filled with pharmaceuticals and/or non-pharmaceuticals, closed, crimped (or closed) and ready to be dispensed, they are optionally inserted in trays located in the pharmaceutical tote module (for example for envelopes) and/or in the storage module (for example for containers). In some embodiments, the trays and/or containers are then picked up by the personnel and dispensed to the patients, and/or optionally by the patients themselves. In some embodiments, the number of trays in the pharmaceutical tote module is from about 1 to about 50. Optionally, the number of trays in the pharmaceutical tote module is from about 4 to about 40. Optionally, the number of trays in the pharmaceutical tote module is from about 6 to about 30. Optionally, the number of trays in the pharmaceutical tote module is from about 12 to about 20. In some embodiments, the number of containers that can be stored in the storage module is from about 1 to about 100. Optionally, from about 1 to about 200. Optionally, from about 1 to about 300. In some embodiments, the envelopes and/or containers filled with pharmaceuticals and/or non-pharmaceuticals are dispensed directly to the user when the user directly accesses the device.

In some embodiments, the pharmaceutical tote module and/or storage module can be disconnected (dismounted) from the frame of the pharmaceutical dispensing machine. In some embodiments, the pharmaceutical tote module and/or storage module can be extracted from the frame of the pharmaceutical dispensing machine without the need to dismount and/or disassemble the other modules.

In some embodiments, this configuration is potentially advantageous during repairs and/or maintenance. In some embodiments, the time required to bring the pharmaceutical dispensing machine to normal functioning is very short (e.g. minutes). In some embodiments, the technician can pull out a module and then insert a new one at its place.

### Exemplary Pharmaceutical Operational Modules Section

In some embodiments, the pharmaceutical operational modules section comprises a plurality of modules, each one responsible for a different operational aspect of the preparation and/or dispensing of the pharmaceuticals. In some embodiments, the pharmaceutical operational modules section comprises, for example, a printer module, a crimper module, a container closing module, an envelope and/or container module and a control module. Optionally, other techniques can be used in the different modules, for example a stapler or gluing instead of crimping. Optionally, in the pharmaceutical operational modules section, spare consumable parts can be stored in order for the machine to use them when necessary. In some embodiments, the envelope and/or container will always be dispensed after it was labeled with the name of the patient.

In some embodiments, each of the modules located in the pharmaceutical operational modules section can be disconnected (dismounted) from the frame of the pharmaceutical operational modules section, without affecting the operation of the rest of the modules. In some embodiments, the envelope modules can be disconnected (dismounted) from the pharmaceutical operational modules section. In some embodiments, the printer module can be disconnected (dismounted) from the pharmaceutical operational modules section. In some embodiments, the crimper module can be disconnected (dismounted) from the pharmaceutical operational modules section. In some embodiments, other backup consumables found are: ink for the printer, staples for the stapler, more envelopes, and other. In some embodiments, the personnel of the facility can replace the consumables of the modules.

In some embodiments, the printer module is responsible for printing on the envelopes and/or containers. In some embodiments, the crimper module is responsible for closing the envelopes after the pharmaceutical have being inserted in them. In some embodiments, the envelope and/or container module is responsible for storing and preparing the envelopes and/or containers before use. In some embodiments, the control module is responsible, for example, for controlling the packaging and dispensing operations (e.g. control of the other modules), communicate with external regional or central server and perform routinely self-diagnostic test to ensure the continuous operation of the pharmaceutical dispensing machine.

Further disclosure of the technology can be found in International Patent Application IB2020/052052, which is hereby incorporated herein by reference in its entirety.

### Exemplary Uses for Modular Pharmaceutical Dispensing Machines

In some embodiments, the modularity of the pharmaceutical dispensing machine can be used for more than just backup and/or redundancy in cases of technical malfunctions. In some embodiments, the modularity of the pharmaceutical dispensing machine is used to respond to demands and operational needs of the specific locations where the machines are located.

In some embodiments, examples of demands and/or needs may be, for example:
Different sizes of dispensing machines: a specific size of machine that can handle and storage a different amount of pharmaceuticals and/or non-pharmaceuticals is provided according to the needs of the place. For example, machines that can handle 150 or 200 or 250 different types of pharmaceuticals and/or non-pharmaceuticals can be provided by adding or removing specific modules.
Increase/Decrease in population: when a specific place suffers either an increase or a decrease in the population that requires the pharmaceutical dispensing service, different modules can be added/removed according to the change.
Multiple modules that perform the same task can have different configurations: for example, a dispensing machine that comprises two different envelope and/or container modules, each one with a different size of envelops and/or containers can work simultaneously. In the case where one of the two sizes is not required, a module can be replaced so the two envelope and/or container modules comprise the same envelope size.
Easy upgradability: modules can be upgraded, either in their hardware or in their software, outside the dispensing machine, and the technician only needs to replace an old module with an upgraded module without the need to replace the whole pharmaceutical dispensing machine.
Easy to transport: separate small modules are transported more easily than whole machines.
Special size pharmaceuticals and/or non-pharmaceutical content: in case a pharmaceutical dispensing machine requires to dispense pharmaceuticals and/or non-pharmaceutical that have a special size, either bigger or smaller than the average pharmaceutical and/or non-pharmaceutical size, a dedicated module/unit/element can be incorporated into the pharmaceutical dispensing machine without the need to replace the whole machine.
Different pharmaceuticals and/or non-pharmaceutical mix and amount of pharmaceuticals and/or non-pharmaceutical requires system adjustment.
Modules can be tested remotely for diagnostics: besides the built-in diagnostic mechanism of the pharmaceutical dispensing machine, the modules can be monitored and/or diagnosed remotely from the main server, without the need to monitor and/or diagnose the whole machine.
Predetermined stock: in some embodiments, pharmaceutical array modules 202 are filled with predetermined stock of pharmaceuticals and/or non-pharmaceuticals, and when in need, are brought into the pharmaceutical dispensing machine and/or the pharmaceutical dispensing vehicle (see below).

In some embodiments, different sizes of pharmaceutical dispensing machines utilize the same modules. In some embodiments, the different modules are interchangeable between pharmaceutical dispensing machines of different sizes.

In some embodiments, the pharmaceutical dispensing machine 104 uses redundancy of the modules, the units and the elements to perform the dispensing process. For example, in the cases where there are two mechanical arms, both mechanical arms are used to transport pharmaceuticals from their container (or multiple redundant containers containing the same and/or different pharmaceuticals) to the envelope (or multiple envelopes in the case where there are more than one envelope module).

### Exemplary Dispensing of Pharmaceuticals and Non-Pharmaceuticals

In some embodiments, it is required that more than one type of pharmaceuticals and/or non-pharmaceuticals is needed to be inserted in one specific envelope and/or container. For example, when a patient needs to receive more than one type of pharmaceutical and/or non-pharmaceutical. Therefore, during the same dispensing event, more than one type of pharmaceutical and/or non-pharmaceutical is inserted in the same transporting container (e.g. an envelope and/or a bottle and/or a container). In some embodiments, patients require medication that are in different form, for example pills and medicine in liquid form. In some embodiments, the pharmaceutical dispensing machine will provide two distinct transporter containers to the same patient, one for example having the pills and the other for example carrying the liquid medicine. In some embodiments, the pharmaceutical dispensing machine will insert all the pharmaceuticals and/or non-pharmaceuticals, pills and liquid medicine in the same transporter container. In these cases, the liquid medicine is first inserted in a small container that is then inserted in the main container together with the rest of the contents. It should be understood that the pharmaceutical dispensing machine dispenses pharmaceuticals that are ready to be taken by the patients. For example, if there are pharmaceuticals that need to be reconstituted by adding water, the reconstitution is performed in the machine itself and then bottled before dispensing it to the patient. In some embodiments, types of pharmaceuticals are selected from one or more of the group consisting of pills, liquids, spray, injections, ointments, creams, patches, and any combination thereof. In some embodiments, inserting in one specific envelope and/or container means that more than one type of pharmaceutical is inserted, for example, in the same envelope. In some embodiments, inserting in one specific envelope and/or container means in a same compartment in a container, for example, a specific container may comprise one or more compartments, and, in some embodiments, one or more distinct pharmaceuticals and/or non-pharmaceuticals are inserted in the same compartment between the one or more compartments, thereby providing a plurality of doses (comprising one or more distinct pharmaceuticals and/or non-pharmaceuticals) to be taken at different times by the same user. In some embodiments, a potential advantage of providing one or more distinct pharmaceuticals and/or non-pharmaceuticals inserted in the same compartment is that it ensures that a user gets all the required pharmaceuticals and/or non-pharmaceuticals. Also, it facilitates the management of the pharmaceutical per user. Also, it reduces the possibility of errors and/or thefts of pharmaceuticals and/or non-pharmaceuticals.

In some embodiments, the envelope and/or container comprises more than one compartment. In some embodiments, a different type of pharmaceuticals and/or non-pharmaceuticals is inserted in a different compartment in the same envelope and/or container. In some embodiments, when pharmaceuticals and/or non-pharmaceuticals are required to be combined with other compounds and/or water, the pharmaceuticals and/or non-pharmaceuticals is inserted in one compartment and the other compounds and/or water are inserted in a different compartment of the same envelope and/or container.

As used herein, in some embodiments, the term “dispensing event” means the same session of preparing said at least one pharmaceutical carrier package for more than one of said patient. In some embodiments, the term “dispensing event” means the actions taken by the machine from the moment the machine begins allocating one or more different and independent from each other ready-to-be-taken-by-a-user pharmaceuticals and/or non-pharmaceuticals from one or more containers until they are inserted into a carrier container. In some embodiments, the term “dispensing event” means the actions taken by the machine from the moment the machine begins allocating one or more different and independent from each other ready-to-be-taken-by-a-user pharmaceuticals and/or non-pharmaceuticals from one or more containers into a carrier container and until the container is ready to be dispensed. In some embodiments, a dispensing event may be a macro-dispensing event comprising a plurality of dispensing events, where each dispensing event is as disclosed above, therefore providing a plurality of containers, each container having one or more compartments and one or more different and independent from each other ready-to-be-taken-by-a-user pharmaceuticals. In some embodiments, a dispensing event may be a mega-dispensing event comprising a plurality of macro-events, therefore providing a massive quantity of pharmaceuticals to be dispensed, to a massive quantity of users at a determined time-period. In some embodiments, at each mega-dispensing event and/or macro-dispensing event and/or a dispensing event, one or more pharmaceutical dispensing machines are used.

In some embodiments, the pharmaceutical dispensing machine is configured to dispense other than pharmaceuticals, in some embodiments also called non-pharmaceuticals, for example, first aid materials like plasters in a variety of different sizes and shapes, disinfectants, bandages, alcohol, tourniquets, small, medium and large sterile gauze dressings, sterile eye dressings, triangular bandages, crêpe rolled bandages, safety pins, disposable sterile gloves, disposable sterile masks, tweezers, scissors, alcohol-free cleansing wipes, sticky tape, thermometer, skin rash cream, such as hydrocortisone or calendula, cream or spray to relieve insect bites and stings, antiseptic cream, painkillers such as paracetamol (or infant paracetamol for children), aspirin (not to be given to children under 16), or ibuprofen, cough medicine, antihistamine cream or tablets, distilled water for cleaning wounds, eye wash and eye bath, pocket mask for CPR, dental kit, sterile knifes/bistoury, syringes and more.

### Exemplary Different Configurations of the Pharmaceutical Dispensing Machine

In some embodiments, due to the modular nature of the pharmaceutical dispensing machine, a plurality of configurations are achieved. For example, the pharmaceutical array module can be located on the upper side of the machine while the pharmaceutical tote module and/or storage module and the pharmaceutical operational modules section, are located at the bottom. In some embodiments, the mechanical arm module is adapted to reach all necessary parts of the machine.

In some embodiments, due to the modular nature of the pharmaceutical dispensing machine, a plurality of configurations are achieved. In some embodiments, the pharmaceutical dispensing machine is located in a room, where the different modules are not included in one specific frame, but are distributed over the room. In some embodiments, the different modules are connected to each other by dedicated arm modules and/or other transportation elements (e.g. conveyors). In some embodiments, when the pharmaceutical dispensing machine is located in a room, the room acts as one specific frame, thereby providing a pharmaceutical dispensing machine with all components required for the pharmaceutical dispensing process, which, in some embodiments, also include backup and/or redundant software and/or hardware. Therefore, in some embodiments, the external envelope and/or frame of the pharmaceutical dispensing machine is a room. Optionally, the external envelope and/or frame of the pharmaceutical dispensing machine is a plurality of rooms. In some embodiments, the different modules located in a plurality of rooms are connected to each other by dedicated arm modules and/or other transportation elements (e.g. conveyors). Optionally, the external envelope and/or frame of the pharmaceutical dispensing machine is a building, optionally comprising a plurality of rooms. In some embodiments, the different modules located in different rooms in the building are connected to each other by dedicated arm modules and/or other transportation elements (e.g. conveyors). In some embodiments, due to the modular nature of the pharmaceutical dispensing machine, modules can be added and/or removed as necessary. For example, additional pharmaceutical array modules can be added to a specific pharmaceutical dispensing machine in times of need, to increase the quantity of pharmaceutical that said pharmaceutical dispensing machine can storage. For example, additional pharmaceutical array modules can be added to a specific pharmaceutical dispensing machine in times of need, to increase the storage capacity of ready to be delivered envelopes and/or containers.

In some embodiments, due to the modular nature of the pharmaceutical dispensing machine, the pharmaceutical dispensing machine can be configured to be a deployable pharmaceutical dispensing machine.

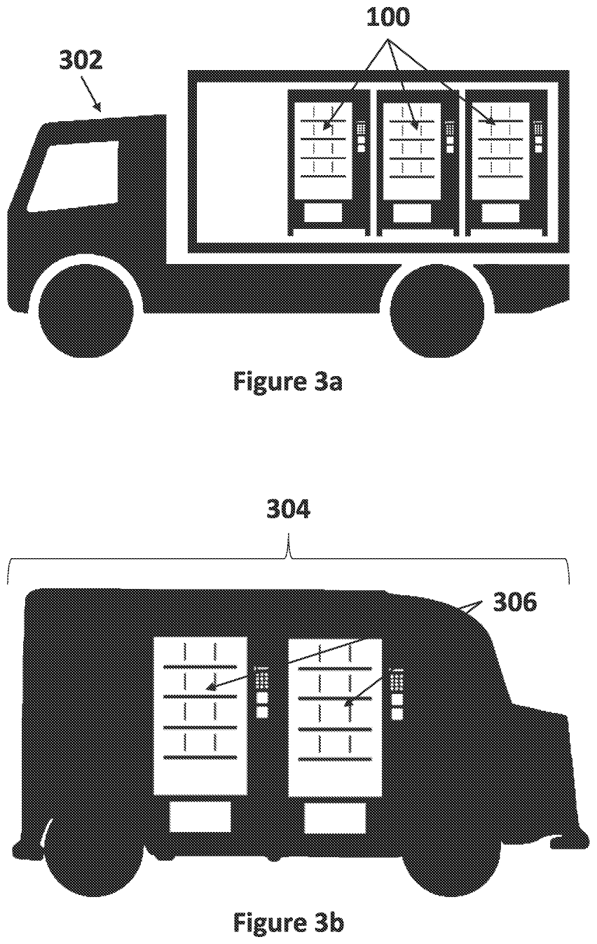

Referring now to FIGS. 3a and 3b, illustrating two exemplary deployable pharmaceutical dispensing machines, according to some embodiments of the invention. In some embodiments, one type of deployable pharmaceutical dispensing machine are one or more mobile pharmaceutical dispensing machines 104 that are configured to be loaded in a truck 302 (or any suitable vehicle), as shown for example in FIG. 3a, and deployed to any location. In some embodiments, the deployment can be for a specific time window, for example a day, a week, a month, or more or less or any interval time window between those. In some embodiments, the truck 302 can carry as many mobile pharmaceutical dispensing machines as necessary for a deployment. In some embodiments, the mobile pharmaceutical dispensing machine are configured to be repeatedly deployed, meaning they are constructed of stronger materials and/or may comprise dedicated attachments and locking mechanisms that facilitate their loading and offloading from trucks.

In some embodiments, another type of deployable pharmaceutical dispensing machine is when a vehicle is configured to be the pharmaceutical dispensing machine 304, as shown for example in FIG. 3b. In some embodiments, the vehicle is modified to allow users to receive pharmaceuticals and/or non-pharmaceuticals from an access panel 306 of the pharmaceutical dispensing machine from outside of the vehicle, while dedicated personnel and/or technicians can access the back of the pharmaceutical dispensing machine, from the inside of the vehicle. The term “pharmaceutical dispensing vehicle” refers hereinafter to a pharmaceutical dispensing machine having one or more wheels which allows the pharmaceutical dispensing machine to move from one location to another, either inside a building or outside, between buildings, and also at least one motor element in communication with the one or more wheels and configured to actively rotate the one or more wheels. In some embodiments, the pharmaceutical dispensing vehicle does not comprise a motor and it is moved between locations by external motorized means (e.g. a truck). In some embodiments, a potential advantage of the pharmaceutical dispensing vehicle is that it can be easily and rapidly deployed to any location.

In some embodiments, the mobile pharmaceutical dispensing machine can optionally comprise mobility mechanisms like the pharmaceutical dispensing vehicle, therefor having one or more wheels which allows the mobile pharmaceutical dispensing machine to move from one location to another, either inside a building or outside, between buildings, and also at least one motor element in communication with the one or more wheels and configured to actively rotate the one or more wheels.

In some embodiments, mobile pharmaceutical dispensing machines and/or pharmaceutical dispensing vehicles differ from known generic dispensing machines in that they provide personalized dispensing services. For example, in generic dispensing machines the contents are pre-packed and/or ready to be dispensed ‘as is’, in the example of sweets, beverages and pre-packed medicines. Contrary to this, the mobile pharmaceutical dispensing machines and/or pharmaceutical dispensing vehicles do not contain pre-arranged packages of pharmaceuticals, rather they prepare personalized packages inside the machines themselves containing the necessary contents to be dispensed to that specific user. In some embodiments, the mobile pharmaceutical dispensing machines and/or pharmaceutical dispensing vehicles do not contain ready to be dispensed boxes of pharmaceuticals and/or non-pharmaceuticals. In some embodiments, the mobile pharmaceutical dispensing machines and/or pharmaceutical dispensing vehicles prepare inside themselves each package that needs to be dispensed. In some embodiments, the principal behind the mobile pharmaceutical dispensing machines and/or pharmaceutical dispensing vehicles is not to replace the human vendor by a robot, rather to replace a human pharmacist with a robot in the preparation of pharmaceuticals and/or non-pharmaceuticals to be dispensed.

Furthermore, contrary to known generic dispensing machines that they display their content, the mobile pharmaceutical dispensing machines and/or pharmaceutical dispensing vehicles do not display their content. In some embodiments, a potential advantage of this is that often pharmaceuticals are light sensitive and having them displayed may damage them.

Additionally, the mobile pharmaceutical dispensing machines and/or pharmaceutical dispensing vehicles can optionally comprise a user interface panel comprising a two-way communication system comprising one or more screens, one or more cameras and one or more microphones, configured to allow communication between the user and an authorized personnel, for example a physician and/or a nurse and/or a pharmacist. In some embodiments, the dispensing of pharmaceuticals and/or non-pharmaceuticals comprises communicating with one or more authorized personnel and performing one or more of revision, verification, authentication of the request of the user. In some embodiments, when the outcome of the reviewing/verifying/authenticating of the request is positive, then the pharmaceuticals and/or non-pharmaceuticals are dispensed. In some embodiments, the reviewing/verifying/authenticating is performed by one or more ways between digital verification, visual verification by means of a video camera (e.g. face recognition), sound (voice) recognition, fingerprint recognition, two-way authentication protocols, etc. In some embodiments, the user interface is accessed by mobile devices, like cellphones, tablets, etc.

### Exemplary Uses for Pharmaceutical Dispensing Vehicles

In some embodiments, pharmaceutical dispensing vehicles can be used for a plurality of scopes, as will be further discussed below.

Providing pharmaceutical dispensing services to field hospitals: in some embodiments, the mobile pharmaceutical dispensing machine and/or the pharmaceutical dispensing vehicle are used to complement field hospitals that require pharmaceutical dispensing services. For example, during a pandemic, like the COVID-19 (Coronavirus Disease 2019) pandemic, patients are ordered to avoid visiting the hospitals and/or physician's office in the case of developing symptoms. Therefore, field hospitals are deployed over central locations over the city to receive and treat patients that are suspected to have contracted COVID-19. In addition to the field hospital, a mobile pharmaceutical dispensing machine and/or a pharmaceutical dispensing vehicle is deployed to the location to provide pharmaceutical and/or non-pharmaceutical dispensing services._In some embodiments, the mobile pharmaceutical dispensing machine and/or the pharmaceutical dispensing vehicle can be accessed only by emergency personnel (e.g. paramedics, doctors, nurses), since its role is to provide pharmaceutical support to the medical team. In some embodiments, a potential advantage for the use of the pharmaceutical dispensing devices of the invention is that is allows the medical personnel to treat a wider variety of problems that could not haven possible to treat due to the limit space in the ambulance and/or paramedic bag. Furthermore, another potential advantage is that it allows the medical personnel to treat the patients at the location and send the patients home without the need to be transported to the hospital.

In some embodiments, the mobile pharmaceutical dispensing machine and/or the pharmaceutical dispensing vehicle can be accessed by emergency personnel (e.g. paramedics, doctors, nurses) and also by random users.

Providing pharmaceutical dispensing services to quarantined locations: in some embodiments, the mobile pharmaceutical dispensing machine and/or the pharmaceutical dispensing vehicle are used to complement temporary quarantined locations that require pharmaceutical dispensing services. For example, in a hotel that is used for the isolation and quarantine COVID-19 (Coronavirus Disease 2019), a mobile pharmaceutical dispensing machine and/or a pharmaceutical dispensing vehicle is deployed to the hotel. The patients and/or dedicated personnel (e.g. physicians, nurses) can access the mobile pharmaceutical dispensing machine and/or the pharmaceutical dispensing vehicle and receive the necessary pharmaceuticals and/or non-pharmaceuticals without risking exposing other people. Same scenario is used for quarantined location in a hospital or any other structure. In some embodiments, the mobile pharmaceutical dispensing machine and/or the pharmaceutical dispensing vehicle can be accessed only by emergency personnel (e.g. paramedics, doctors, nurses), since its role is to provide pharmaceutical support to the medical team. In some embodiments, a potential advantage for the use of the pharmaceutical dispensing devices of the invention is that is allows the medical personnel to treat a wider variety of problems that could not haven possible to treat due to the limit space in the ambulance and/or paramedic bag. Furthermore, another potential advantage is that it allows the medical personnel to treat the patients at the location and avoiding to send the patients to the hospital.

In some embodiments, the mobile pharmaceutical dispensing machine and/or the pharmaceutical dispensing vehicle can be accessed by emergency personnel (e.g. paramedics, doctors, nurses) and also by the patients located at the quarantined location.

Scheduled events: in some embodiments, pharmaceutical dispensing vehicles are sent to scheduled events, for example concerts, sport events, political and non-political manifestations and more. In some embodiments, the pharmaceutical dispensing vehicle is equipped with predetermined pharmaceuticals and/or non-pharmaceuticals according to:
The type of scheduled event (e.g. concert, sport event);
The type of population that will attend the event (e.g. young population, elderly population, mixed population);
The location of the event (e.g. stadium, city roads, park, etc.);

In some embodiments, once an event is scheduled, the pharmaceutical dispensing system analyzes all the data related to the event and prepares the pharmaceutical array modules 202 destined to be loaded on the mobile pharmaceutical dispensing machine and/or the pharmaceutical dispensing vehicle. In some embodiments, in addition, people that know that will attend the event can request from the pharmaceutical dispensing system to load specific pharmaceuticals and/or non-pharmaceuticals into the mobile pharmaceutical dispensing machine and/or the pharmaceutical dispensing vehicle that will be deployed at the event. In some embodiments, a potential advantage of this is that it allows flexibility of dispensing to all users. In some embodiments, the mobile pharmaceutical dispensing machine and/or the pharmaceutical dispensing vehicle can be accessed only by emergency personnel (e.g. paramedics, doctors, nurses) during the scheduled event, since its role is to provide pharmaceutical support to the medical team. In some embodiments, a potential advantage for the use of the pharmaceutical dispensing devices of the invention is that is allows the medical personnel to treat a wider variety of problems that could not haven possible to treat due to the limit space in the ambulance and/or paramedic bag. Furthermore, another potential advantage is that it allows the medical personnel to treat the patients at the location of the event and send the patients home without the need to be transported to the hospital.

In some embodiments, the mobile pharmaceutical dispensing machine and/or the pharmaceutical dispensing vehicle can be accessed by emergency personnel (e.g. paramedics, doctors, nurses) and also by random users during the scheduled event.

Emergency events: in some embodiments, pharmaceutical dispensing vehicles are sent to emergency events, for example shootings, plagued locations, accidents, explosions, fires, quarantined locations and more. In some embodiments, the pharmaceutical dispensing vehicle is equipped with predetermined pharmaceuticals and/or non-pharmaceuticals according to:
The type of emergency event (e.g. shootings, plagued locations, accidents, explosions, fires, quarantined locations);
The type of population that is affected by the emergency (e.g. young population, elderly population, mixed population);
The location of the event (e.g. school, hospitals, stadium, city roads, parks, commercial centers, etc.).

Mobile pick-up location of pharmaceuticals: in some embodiments, pharmaceutical dispensing vehicles are sent to be pick-up locations of pharmaceuticals for the general population, for example a user can request to pick up a determined pharmaceutical from the pharmaceutical dispensing vehicle instead of going to a regular pharmacy. In some embodiments, the pharmaceutical dispensing vehicle is equipped with the predetermined pharmaceuticals and/or non-pharmaceuticals required by the user. In some embodiments, the user can schedule the window and location where the pick-up will take place. In some embodiments, the user can request which pharmaceuticals and/or non-pharmaceuticals will be dispensed by the pharmaceutical dispensing vehicle. In some embodiments, the pharmaceutical dispensing vehicle is deployed to the address of the homes of the users to deliver pharmaceuticals and/or non-pharmaceuticals. In some embodiments, the patient schedules the home-delivery of the pharmaceuticals and/or non-pharmaceuticals with the system and the pharmaceutical dispensing vehicle arrives at the home address of the patient.

### General Information Regarding Mobile Pharmaceutical Dispensing Devices and Pharmaceutical Dispensing Vehicles

In some embodiments, the mobile pharmaceutical dispensing machine and or the pharmaceutical dispensing vehicle comprise one or more built-in communication module configured to allow connection of the mobile pharmaceutical dispensing machine and or the pharmaceutical dispensing vehicle to the server, and therefore, to the information required for the pharmaceutical dispensing process. In some embodiments, the mobile pharmaceutical dispensing machine and or the pharmaceutical dispensing vehicle comprise at least one back-up communication module (e.g. satellite communication software and hardware), configured to be activated in case the built-in communication elements cannot operate. In some embodiments, dedicated personnel (e.g. physicians, pharmacists, etc.) can connect with the mobile pharmaceutical dispensing machine and or the pharmaceutical dispensing vehicle and remotely request the dispensing of pharmaceuticals and/or non-pharmaceuticals.

In some embodiments, the mobile pharmaceutical dispensing machine and or the pharmaceutical dispensing vehicle is equipped with identification hardware and software configured to recognize dedicated personnel (e.g. pharmacists, physicians, nurses) and dispense pharmaceuticals and/or non-pharmaceuticals on demand. For example, during an emergency event, the pharmaceutical dispensing vehicle is able to positively identify a paramedic and, on demand, dispense what is requested from the paramedic. In some embodiments, the requests and/or identification is performed using one or more of: an identification document (e.g. card), face recognition technology, voice recognition technology, dedicated wireless hardware (e.g. cellphones, tablets, voice activated hardware like Alexa).

In some embodiments, the mobile pharmaceutical dispensing machine and or the pharmaceutical dispensing vehicle is equipped with identification hardware and software configured to recognize the patients and/or dedicated personnel trying to access the services of said pharmaceutical dispensing vehicle and allow dispensing of the pharmaceuticals and/or non-pharmaceuticals. In some embodiments, the identification is performed using one or more of: an identification document (e.g. card), face recognition technology, voice recognition technology, dedicated wireless hardware (e.g. cellphones, tablets, voice activated hardware like Alexa). Furthermore, in some embodiments, the mobile pharmaceutical dispensing machine and or the pharmaceutical dispensing vehicle is equipped with written prescription processing system comprising hardware configured to read and/or scan and/or identify written prescriptions presented by patients, communicate with the server to assess the written prescription and dispense what is requested when is approved. In addition, in some embodiments, the mobile pharmaceutical dispensing machine and or the pharmaceutical dispensing vehicle is equipped with hardware configured to allow payments locally at the mobile pharmaceutical dispensing machine and or the pharmaceutical dispensing vehicle. Lastly, in some embodiments, the user interface is configured to display instructions on how to use and/or consume the pharmaceuticals and/or non-pharmaceuticals.

In some embodiments, the mobile pharmaceutical dispensing machine and or the pharmaceutical dispensing vehicles are equipped with self-sterilization equipment configured to sterilize the parts that are in contact with the patients. In some embodiments, exemplary self-sterilization equipment is UV lamps, high temperature air and/or vapor, or any combination thereof. In some embodiments, a potential advantage of this sterilization equipment is that it reduces the chances to spread diseases between patients that utilize the mobile pharmaceutical dispensing machine and or the pharmaceutical dispensing vehicle.

In some embodiments, the pharmaceutical dispensing vehicle is configured so a user interface panel and a delivery window, both part of a user interface module partially located inside the vehicle and/or the machine, are located at a distance of from about 0.9 meters to about 1.7 meters from the ground, thereby allowing a person standing and/or sitting in a wheelchair outside the pharmaceutical dispensing vehicle to easily access the user interface panel and the delivery window.

In some embodiments, the delivery window can be an actual window that opens, or can be an aperture, or any other known in the art delivery access points.

In some embodiments, during the operation of the mobile pharmaceutical dispensing machine and or the pharmaceutical dispensing vehicle, real-time updates are sent to a server. In some embodiments, the updates are used to monitor the requirements of pharmaceuticals and/or non-pharmaceuticals, and predict the near future requirements of the same.

In some embodiments, the user interface comprises a two-way communication system comprising one or more screens, one or more cameras and one or more microphones, configured to allow communication between the user and an authorized personnel, for example a physician and/or a nurse and/or a pharmacist. In some embodiments, the dispensing of pharmaceuticals and/or non-pharmaceuticals comprises communicating with one or more authorized personnel and reviewing the request of the user. In some embodiments, when the outcome of the reviewing of the request is positive, then the pharmaceuticals and/or non-pharmaceuticals are dispensed.

In some embodiments, predetermined stocks of pharmaceutical array modules 202 are prepared and optionally stored with predetermined stock of pharmaceuticals and/or non-pharmaceuticals, and when in need, are brought into the mobile pharmaceutical dispensing machine or the pharmaceutical dispensing vehicle. In some embodiments, predetermined stocks are prepared according to the types of events (see types of event above). In some embodiments, the predetermined stocks are kept in storage, waiting for the right event to occur. For example, predetermined stocks filled with relevant pharmaceuticals and non-pharmaceuticals adapted to be used in case of a terrorist attack are prepared and kept in storage. When a terrorist attack occurs, the pharmaceutical array modules are inserted in the pharmaceutical dispensing vehicles and sent to the place of the attack.

### Exemplary Automation of Mobile Pharmaceutical Dispensing Machines and Pharmaceutical Dispensing Vehicles

In some embodiments, the mobile pharmaceutical dispensing machine and/or the pharmaceutical dispensing vehicle are autonomous mobile pharmaceutical dispensing machines and/or pharmaceutical dispensing vehicles. In some embodiments, the machines and/or vehicles comprise autonomous mobility technology which allows them to autonomously move from one location to another. In some embodiments, the locations can be rooms inside a building, like rooms in a hospital or rooms in a hotel. In some embodiments, locations can be different addresses of places, like buildings, houses, commercial centers, hospitals, etc. In some embodiments, the autonomous mobile pharmaceutical dispensing machine and/or the pharmaceutical dispensing vehicle comprise location and navigation software and hardware which allows the autonomous mobile pharmaceutical dispensing machine and/or the pharmaceutical dispensing vehicle to know their own locations, to know the locations that need to reach and the best way to arrive from one point to the next. Autonomous navigation technologies used are similar to those described in: Faiza Gul, Wan Rahiman & Syed Sahal Nazli Alhady, (2019), A comprehensive study for robot navigation techniques, Cogent Engineering, 6:1, 1632046.

Optionally, the mobile pharmaceutical dispensing machine comprises a robotic helper configured to pick-up ready to be dispensed containers from the mobile pharmaceutical dispensing machine and performed personal deliveries in the vicinity of the mobile pharmaceutical dispensing machine. For example, the mobile pharmaceutical dispensing machine autonomously moves in a hotel used to quarantine COVID-19 positive patients, together with the robotic helper. When they arrive at a certain floor, the robotic helper received from the mobile pharmaceutical dispensing machine all the containers that need to be dispensed in the floor. Then, the robotic helper moves from room to room delivering the containers. Then the mobile pharmaceutical dispensing machine and the robotic helper move to the next floor.

In some embodiments, the autonomous mobile pharmaceutical dispensing machine and/or the pharmaceutical dispensing vehicle comprise a user interface comprising a two-way communication system/hardware optionally comprising one or more screens, one or more cameras and one or more microphones, configured to allow communication between the user and an authorized personnel, for example a physician and/or a nurse and/or a pharmacist. In some embodiments, the dispensing of pharmaceuticals and/or non-pharmaceuticals comprises communicating with one or more authorized personnel and reviewing the request of the user. In some embodiments, when the outcome of the reviewing of the request is positive, then the pharmaceuticals and/or non-pharmaceuticals are dispensed.

### Exemplary Methods

In some embodiments, the one or more of the following actions are performed when activating a mobile pharmaceutical dispensing device and/or vehicle.

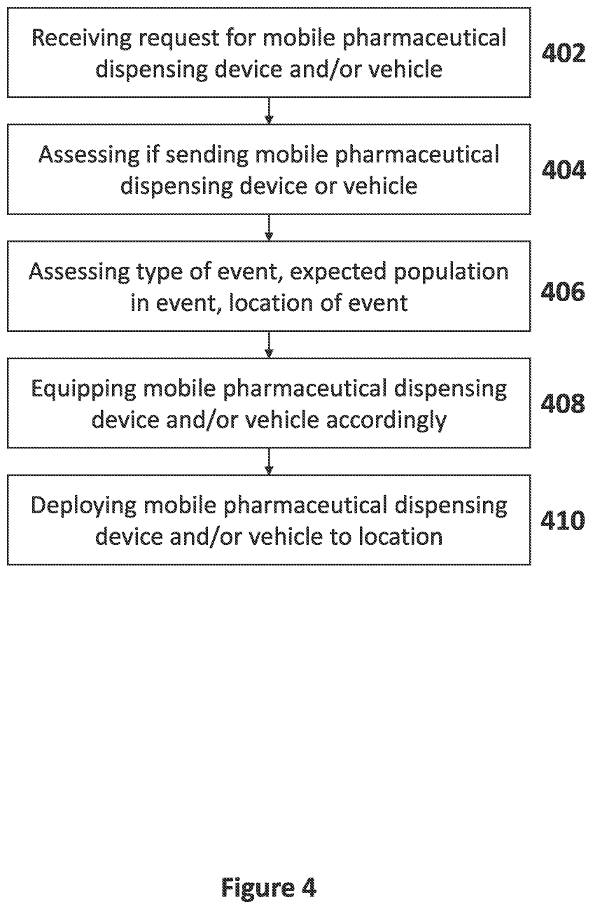

Referring now to FIG. 4, showing a flowchart of an exemplary method of deployment of a pharmaceutical dispensing machine/vehicle for a scheduled event, according to some embodiments of the invention. In some embodiments, a request for a mobile pharmaceutical dispensing device and/or vehicle is received 402. In some embodiments, it is then assessed whether to send a mobile pharmaceutical dispensing machine or a pharmaceutical dispensing vehicle 404. In some embodiments, it is then assessed the parameters of the event 406, for example the type of event, the expected population in event, the location of event, etc. In some embodiments, then the mobile pharmaceutical dispensing machine and/or the pharmaceutical dispensing vehicle are equipped with the necessary pharmaceuticals and/or non-pharmaceuticals 408. In some embodiments, then the mobile pharmaceutical dispensing machine and/or the pharmaceutical dispensing vehicle are deployed 410.

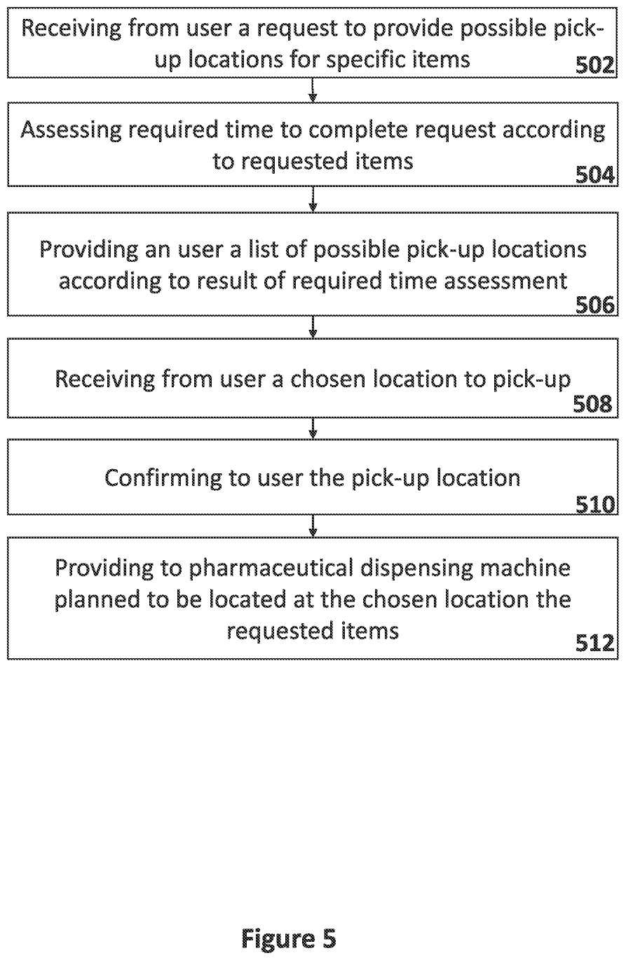

Referring now to FIG. 5, showing a flowchart of an exemplary method of interaction between the deployable pharmaceutical dispensing system and a user, according to some embodiments of the invention. In some embodiments, the pharmaceutical dispensing system is contacted by a user requesting to provide possible and/or available pick-up locations for specific items 502. In some embodiments, the specific items can be pharmaceuticals and/or non-pharmaceuticals. In some embodiments, the system assesses the time required for the different parts of the system to provide the specific items 504, for example, how much time will take to move the requested items from one storage facility to another, how much time will take to provide the requested item to the specific pharmaceutical dispensing machine that will be deployed to a location, the time in advance that the items need to be already in the specific pharmaceutical dispensing machine before the events located in the database of the system, etc. In some embodiments, after the system has calculated the time required to complete the request according to the specific items, the system provides the user a list of possible pick-up locations 506. In some embodiments, the user then choses a location and the system receives the choice of the chosen location from the user 508. In some embodiments, the system then confirms to the user the pick-up location of the requested items 510. In some embodiments, the system then activates a series of instructions over the different parts of the system to provide the specific pharmaceutical dispensing machine destined to be deployed to the specific location chosen by the user the requested items 512. In some embodiments, optionally, the user may choose to stop the request for any reason, for example, the list of possible locations is not suitable.

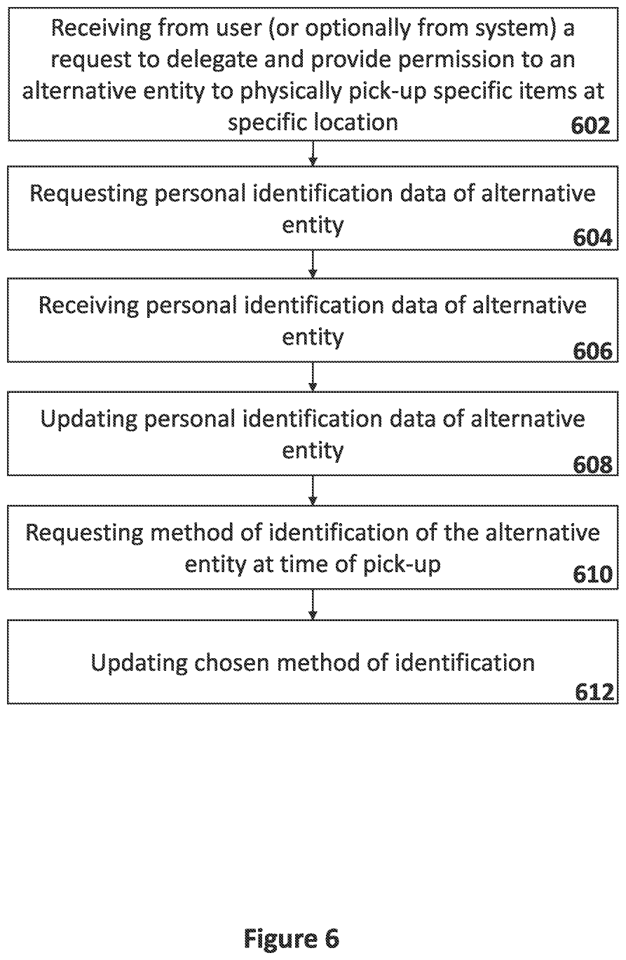

Referring now to FIG. 6, showing a flowchart of an exemplary method of interaction between the deployable pharmaceutical dispensing system and a user, according to some embodiments of the invention. In some embodiments, when, during and/or after a user requests to set up a pick-up location for specific items, the user, and optionally the system, may request to delegate the physical pick-up to another person and/or entity 602. In some embodiments, the system then requests the personal identification data of new entity 604. In some embodiments, the system then receives the personal identification data of the new entity 606. In some embodiments, the system then updates the personal identification data of the new entity 608. In some embodiments, the system then requests a method of identification of the new entity at the time of the pick-up 610. In some embodiments, methods of identification can be for example, an identification card, a dual-identification process using a mobile device, facial recognition, etc. In some embodiments, the system then updates the databases with the chosen method of identification of the new entity 612.

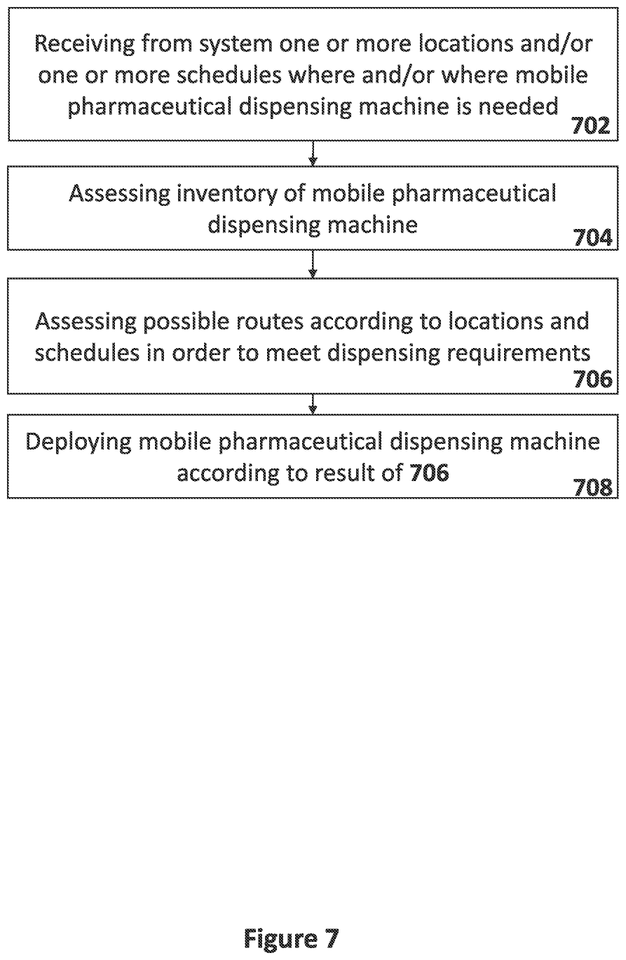

Referring now to FIG. 7, showing a flowchart of an exemplary method of deployment of an autonomous mobile pharmaceutical dispensing machine, according to some embodiments of the invention. In some embodiments, the autonomous pharmaceutical dispensing machine receives from the system one or more locations and/or one or more schedules where and/or where the autonomous mobile pharmaceutical dispensing machine is needed 702. In some embodiments, the location can be close to each other. In some embodiments, the locations can be far from each other. In some embodiments, the locations can be different rooms on a floor in a quarantined location, for example, a hotel that is used to quarantine COVID-19 positive patients. In some embodiments, the autonomous pharmaceutical dispensing machine assesses the inventory to see that it has all the required pharmaceuticals and/or non-pharmaceuticals for the route that will be performed 704. In some embodiments, when some item is missing from the inventory, the autonomous pharmaceutical dispensing machine contacts the server and requests the missing item. In some embodiments, when the missing item cannot be provided the system provides independent means to the missing item. In some embodiments, the autonomous pharmaceutical dispensing machine assesses possible routes according to locations and schedules in order to meet dispensing requirements 706. In some embodiments, a plurality of routes are planned simultaneously. In some embodiments, the route can change during the route and the autonomous pharmaceutical dispensing machine adapts the route in real-time. In some embodiments, the autonomous pharmaceutical dispensing machine is deployed according to the route chosen in the previous assessment 708.

In some embodiments, picking up a pharmaceutical and/or a non-pharmaceutical from a mobile pharmaceutical dispensing machines and/or a pharmaceutical dispensing vehicles is performed only by the patient itself. In some embodiments, picking up a pharmaceutical and/or a non-pharmaceutical from a mobile pharmaceutical dispensing machines and/or a pharmaceutical dispensing vehicles can be performed also by other than the patient itself. For example, in a quarantined hotel, a person from a specific floor can pick up the packages from everybody located in the same floor. In some embodiments, a potential advantage of this is the reduction of people walking around the hotel and potentially reducing the possibility of infecting others. Also, this could help those patient that cannot or it is difficult for them to stand up due to their condition.

It is expected that during the life of a patent maturing from this application many relevant mobile pharmaceutical dispensing devices/vehicles will be developed; the scope of the terms used herein are intended to include all such new technologies a priori.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A mobile pharmaceutical dispensing machine, comprising:
a. an external frame comprising an inside and an outside;
b. one or more pharmaceutical dispensing modules located in said inside of said external frame and configured to perform parts of a pharmaceutical dispensing process of a pharmaceutical dispensing request from at least one user, said pharmaceutical dispensing process comprising moving one or more items from general containers into one or more personalized pharmaceutical dispensing containers;
c. one or more delivery windows facing said outside of said external frame and configured to receive and allow delivery of said one or more personalized pharmaceutical dispensing containers from said one or more pharmaceutical dispensing modules;
d. one or more user interfaces in communication with said one or more pharmaceutical dispensing modules and configured to receive one or more inputs related to said pharmaceutical dispensing request from said at least one user.

2. The mobile pharmaceutical dispensing machine of claim 1, wherein said one or more modules are selected from the group consisting of: at least one pharmaceutical array module, at least one storage module, at least one pharmaceutical operational modules section, at least one user interface module, at least one communication module and at least one mechanical arm module.

3. The mobile pharmaceutical dispensing machine of claim 1, wherein said at least one user interface module is accessed by said at least one user using at least one mobile device.

4. The mobile pharmaceutical dispensing machine of claim 1, wherein said at least one user interface module comprises at least one user interface panel facing said outside of said mobile pharmaceutical dispensing machine and located at a distance from about 0.9 meters to about 1.7 meters from the ground on which said mobile pharmaceutical dispensing machine is standing.

5. The mobile pharmaceutical dispensing machine of claim 1, wherein said at least one user interface module comprises identification hardware and software configured to recognize said at least one user trying to access the services of said mobile pharmaceutical dispensing machine.

6. The mobile pharmaceutical dispensing machine of claim 4, wherein said at least one user interface panel comprises self-sterilization equipment.

7. The mobile pharmaceutical dispensing machine of claim 1, wherein said one or more delivery windows comprises self-sterilization equipment.

8. The mobile pharmaceutical dispensing machine of claim 1, wherein said one or more modules are configured to be independently dismounted from said mobile pharmaceutical dispensing machine; and wherein dismounting said one or more modules does not interrupt said parts of said pharmaceutical dispensing process.

9. The mobile pharmaceutical dispensing machine of claim 1, wherein said one or more user interfaces comprises one or more two-way communication systems comprising one or more screens, one or more cameras and one or more microphones, configured to allow communication between said at least one user and an authorized personnel.

10. The mobile pharmaceutical dispensing machine of claim 1, further comprising at least one written prescription processing system configured to process written prescriptions presented by said at least one user.

11. The mobile pharmaceutical dispensing machine of claim 10, wherein said one or more user interfaces are further configured to display instructions related to said pharmaceuticals.

12. The mobile pharmaceutical dispensing machine of claim 1, wherein said mobile pharmaceutical dispensing machine further comprises one or more wheels and at least one motor in communication with said one or more wheels and configured to actively rotate said one or more wheels.

13. The mobile pharmaceutical dispensing machine of claim 1, wherein said mobile pharmaceutical dispensing machine is an autonomous mobile pharmaceutical dispensing machine and further comprises location and navigation systems.

14. A method for providing a pharmaceutical dispensing vehicle and/or a mobile pharmaceutical dispensing machine to an event, comprising:
a. receiving request for said pharmaceutical dispensing vehicle and/or said mobile pharmaceutical dispensing machine;
b. assessing whether sending said mobile pharmaceutical dispensing machine;
c. assessing one or more parameters of said event;
d. equipping said mobile pharmaceutical dispensing machine according to the results of said assessments;
e. deploying said mobile pharmaceutical dispensing machine to a location of said event.

15. The method according to claim 14, wherein said assessing one or more parameters of said event comprises one or more of assessing type of event, assessing expected population in event, assessing said location of event.

16. The method according to claim 15, wherein said type of event is an emergency event.

17. The method according to claim 16, wherein said emergency event is a viral pandemic event.

18. The method according to claim 15, wherein said type of event is a scheduled event.

19. The method according to claim 15, wherein said expected population comprises one or more of: elderly population, young population, healthy population, sick population, COVID-19 positive population, quarantined population, newborn population, middle age population and mixed population.

20. The method according to claim 14, wherein said location comprises one or more of: public location, private location, a concert, a stadium, a hotel, a hotel used for quarantined patients, a hotel used for COVID-19 quarantined patients, a park, middle of a street, a location of an emergency event and a location of an accident.
